(19) United States
(12) Patent Application Publication
(10) Pub. No.: US 2005/0113759 A1
(43) Pub. Date: May 26, 2005

(54) Catheter retainer

(76) Inventors: Richard L. Mueller JR., Jackson, WY (US); James Kermode, Los Altos, CA (US); Douglas Murphy-Chutorian, Palo Alto, CA (US)

Correspondence Address: OLSON & HIERL, LTD., 36th Floor, 20 North Wacker Drive, Chicago, IL, 60606, US

(21) Appl. No.: 10/720,889

(22) Filed: Nov. 24, 2003

(51) Int. Cl. A61M 5/32

(52) U.S. Cl. 604/174

(57) ABSTRACT

A catheter retainer includes a spacer and an anchor that coact to maintain a catheter in a predetermined position within a mammary duct during a ductal lavage procedure. The spacer and the anchor operate together to resist angular or axial displacement of the catheter. A catheter retainer kit is disclosed as well.

### TECHNICAL FIELD OF THE INVENTION

The invention relates to a catheter retainer suitable for use during a ductal lavage procedure. In particular, the catheter retainer is suitable for substantially maintaining a predetermined position of a catheter within a mammary duct during a ductal lavage procedure.

### BACKGROUND OF THE INVENTION

Breast cancer is one of the health threats most feared by women, and is a common form of cancer in women. In rare instances the human male may also have occurrences of breast cancer. A key to treatment of breast cancer is early detection. For example, an annual mammogram is a method that has been used in hopes of early detection of breast cancer. One problem with mammography is that such an imaging technique can only find breast cancer once it has taken form. All too often, breast cancer is discovered at a stage that is too far advanced, when therapeutic options and survival rates are severely limited. As such, more sensitive and reliable methods and devices are needed to detect cancerous, pre-cancerous, and other cancer markers of the breast at an early stage. Such methods and devices could significantly improve breast cancer survival.

Some methods of detecting breast cancer are based on the fact that a vast majority of instances of breast cancer begins in the lining of mammary ducts. Studies have shown that fluid within the mammary duct contains high levels of breast cancer markers, and that an estimated 80%-90% of all breast cancers occur within the intraductal epithelium of the mammary glands. Fluid within the breast ducts contains an assemblage and concentration of hormones, growth factors and other potential markers comparable to those secreted by, or acting upon, the surrounding cells of the alveolar-ductal system. Likewise, mammary duct fluid typically contains cells and cellular debris, or products that can also be used in cytological or immunological assays. As such, various methods for collection of mammary duct fluid and cellular debris therein have been developed.

One such method is to collect exudate from a mammary duct either when the duct is discharging liquid on its own, or by massaging the breast to cause it to discharge liquid. A problem with such a method is that one has to wait for the mammary duct to discharge liquid or hope for an adequate amount of liquid to be present in the mammary duct.

To enable practitioners to increase the likelihood of obtaining cytological liquid samples from a mammary duct, and to provide control over when such samples may be harvested, a procedure known as ductal lavage has been utilized. This method comprises the introduction of a rinsing solution, such as a saline solution or the like, into a mammary duct, followed by the collection of the introduced solution along with any cells and cellular debris from the mammary duct. Typically, a catheter or cannula having an internal lumen is used to introduce the solution into the mammary duct. Conventional catheters and cannulas include distal portions that may be introduced into a mammary duct via a nipple orifice terminating at the surface of a human nipple, and advanced to a predetermined position with the assistance of an internally positioned dilator. The distal end portion of a catheter is usually only guided into the mammary duct to a depth of approximately 3 to 5 millimeters to optimize the potential for obtaining a cytological liquid sample and to avoid inadvertently lodging the open distal end of the catheter in a mammary duct wall.

In order to harvest or collect the cytological liquid from the mammary duct, several methods have been employed. One is to provide a vacuum source, such as a syringe, to the proximal end of the catheter to draw out the cytological fluid. Another method is to massage the breast to coax liquid within the duct towards the nipple orifice. Due to the relatively shallow depth to which the catheter is inserted into the mammary duct, the methods for harvesting the cytological liquid sometimes result in dislodgement of the catheter from the mammary duct. For example, with the vacuum method the tubing or syringe of the vacuum source can be relatively heavy and cause the catheter to angle away from the preferred substantially perpendicular relationship to the nipple, and can even pry or pull the catheter distal end portion from the duct. The catheter may also be moved deeper into the mammary duct such that the open distal end of the catheter is undesirably buried in a wall of the mammary duct or is moved beyond the desired region in the mammary duct for collecting the liquid sample, which is proximate the sinus. For example with a vacuum source, such as a syringe, the catheter may be inadvertently pushed further into the mammary duct as liquid is introduced to the duct by the syringe as is typical in ductal lavage procedure. In either event, adjustments to the position of the catheter or reinsertion must be performed. With the massaging of the breast, the manual manipulation of the breast often causes the catheter to become dislodged.

A common method of reducing the likelihood of a general catheter dislodging from a patient is to tape the catheter down flat with the patient's skin. Such a method, however, is unsuitable for mammary duct exudate collection procedures, such as ductal lavage, because the catheter cannot be maintained at a substantially perpendicular angle to the nipple surface. Taping the catheter down flat to the patient's skin does not allow a substantially perpendicular angle to be maintained.

Another conventional method of securing the catheter to the patient is to secure strips of tape to the catheter and to the patient while maintaining a perpendicular alignment of the catheter with the nipple surface. The problem with this particular method is that, as discussed, the catheter may still be accidentally pushed deeper into the mammary duct because the strips of tape only limit the proximal movement of the catheter out of the mammary duct, but not further into the duct. The present invention addresses these shortcomings.

### SUMMARY OF THE INVENTION

A catheter retainer is provided which is suitable for maintaining a predetermined position of a catheter into a mammary duct during a ductal lavage procedure. The catheter retainer includes a spacer which is positionable about a catheter inserted into a mammary duct. The spacer is preferably flexible and removably positioned about the catheter after the catheter is introduced into the mammary duct. An anchor, which is operatively engageable with the catheter is also removably securable to the patient's breast. The flexible spacer and the anchor coact together to substantially maintain the alignment and depth of the catheter in the mammary duct and substantially limit the movement of the catheter axially as well as angularly.

Preferably, the spacer is also compressible. For example, the spacer may be made of a compressible elastomer, such as an open cell foamed elastomer or a closed cell elastomer, e.g., a polyurethane foam. Alternatively, the spacer may comprise a spring-type member, such as a leaf spring assembly made from a flexible, resilient plastic material such as polyethylene. The anchor preferably includes an adhesive for securing the anchor to the patient's skin. Since the anchor is engageable with the catheter, the catheter is also secured in position to the patient at the same time.

The anchor and the flexible spacer cooperatively restrain movement of the catheter both laterally and axially to substantially maintain the desired angle and depth of the catheter during the ductal lavage procedure. The catheter retainer of the present invention also provides hands-free securement and positioning of the catheter in order for the practitioner to have greater freedom to perform tasks, such as manipulation of the breast without also having to hold on to the catheter.

The catheter retainer may also be provided in a kit that includes items such as a catheter, a dilator, a nipple illumination cup, an obturator, syringe or a squeeze bulb, and instructional materials.

### BRIEF DESCRIPTION OF THE DRAWINGS

In the drawings,

FIG. 1 is a side view of a prior art catheter retainer with a catheter introduced into a mammary duct of a human breast, which is shown in cross section;

FIG. 2 is another view of the prior art catheter retainer of FIG. 1 showing the catheter displaced angularly or laterally;

FIG. 3 is another view of the prior art catheter retainer of FIG. 1 showing the catheter displaced axially in a distal direction;

FIG. 4 is an exploded perspective view of one preferred embodiment of the present invention showing a catheter retainer together with a catheter;

FIG. 5 is a side view, shown partially in section, showing the catheter position within a mammary duct and the retainer of FIG. 4 positioned about the catheter;

FIG. 6 is a perspective view of an alternate embodiment of the present invention showing a catheter retainer wherein the spacer and anchor are integral;

FIG. 7 is an exploded perspective view of yet another alternate embodiment of the present invention showing a catheter retainer wherein the spacer comprises a leaf spring assembly;

FIG. 8 is an exploded perspective view of still another alternate embodiment of the present invention showing a catheter retainer; and

FIG. 9 is a perspective schematic view of a kit embodying the present invention.

### DESCRIPTION OF PREFERRED EMBODIMENTS OF THE INVENTION

The invention disclosed herein is, of course, susceptible of being embodied in many different devices. Shown in the drawings and described herein below in detail are preferred embodiments of the invention. It is to be understood, however, that the present disclosure is an exemplification of the principles of the invention and does not limit the invention to the illustrated embodiments.

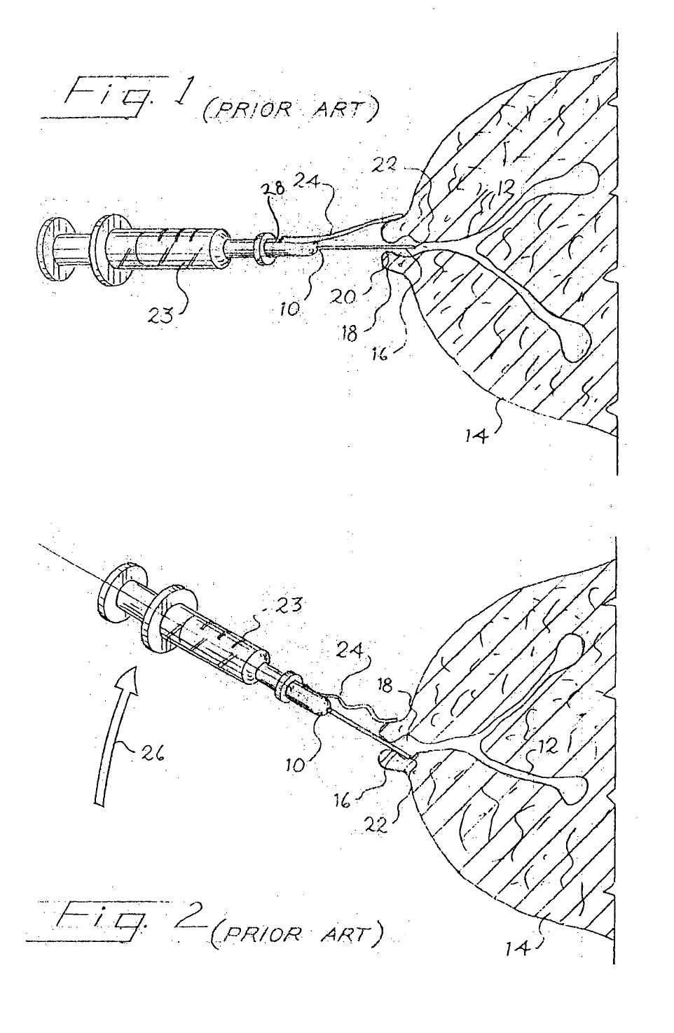

A prior art device for retaining a catheter introduced into a mammary duct during a ductal lavage procedure is shown in FIG. 1. A catheter 10 is introduced into mammary duct 12 in human breast 14. Catheter 10 is inserted to a depth such that the distal end portion 16 of the catheter is proximate to the sinus 18, which is located distal to the sphincter muscle 20. Cytological liquid is urged into the open distal end 22 of the catheter 10 through application of a vacuum source, such as a syringe 23 or the like. The breast 14 may also be massaged by the practitioner to urge the cytological liquid towards the nipple orifice in a manner similar to that performed by lactating women when expressing breast milk.

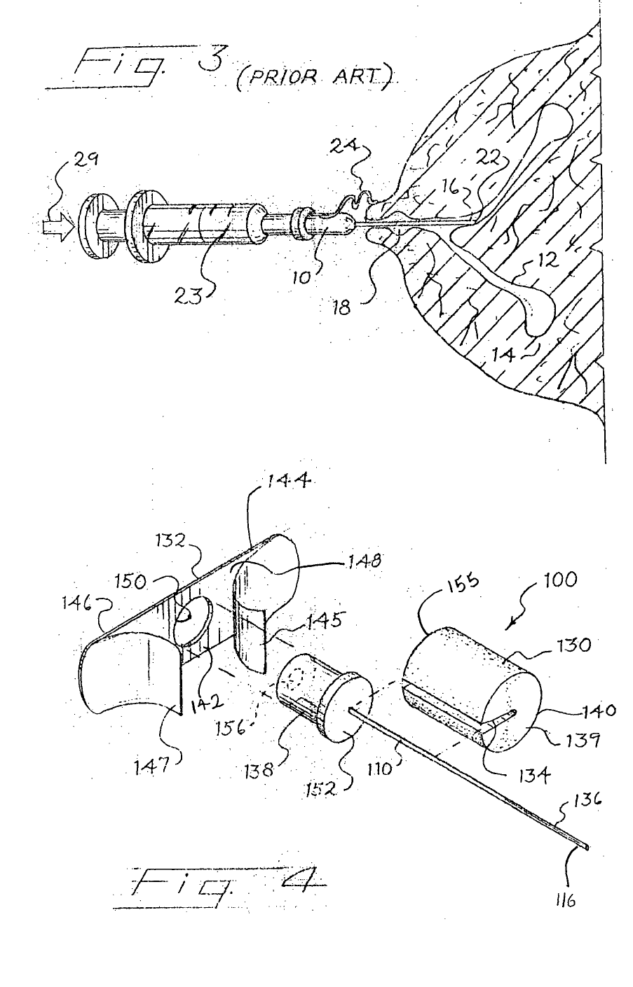

The catheter 10 is secured to the patient's breast 14 with an anchor, such as tape strip 24 which is also secured to the proximal end 28 of catheter 10. Some shortcomings of this prior art retainer are shown in FIGS. 2 and 3. The tape strip 24 serves to provide some resistance to the catheter 10 disengaging from the mammary duct by resisting axial movement of the catheter 10 in a proximal direction which may result in accidentally pulling the catheter 10 away from the patient. However, the tape strip 24 offers little or no resistance to angular displacement of catheter 10 as shown in FIG. 2. As a result, a lateral or angular force, such as depicted as arrow 26, will cause the tape strip 24 to deform and allow the distal end portion 16 of catheter 10 to possibly contact or penetrate the wall of the mammary duct 12. Such contact with the mammary duct 12 by the open distal end 22 of the catheter 10 will reduce or prevent the harvesting of cytological liquid by blocking the open end 22, and may also cause damage to the mammary duct wall. Similar misalignment of the catheter 10 may also be caused by movement of patient or patient's breast during the procedure. Also, although not shown, if the angular displacement is significant enough, the distal end portion of the catheter 10 may be inadvertently pried out of the mammary duct.

Tape strip 24 also does not provide any resistance against a force on the catheter such that would cause the catheter to penetrate deeper into the mammary duct as shown in FIG. 3 as force arrow 29. Such a change in depth of the catheter often results in the distal end 22 of the catheter 10 contacting a duct wall and resulting in the problems discussed above. Alternatively, the change in depth can also move the open distal end 22 away from the optimal position proximate the sinus 18 such that the amount of cytological liquid harvested is lessened.

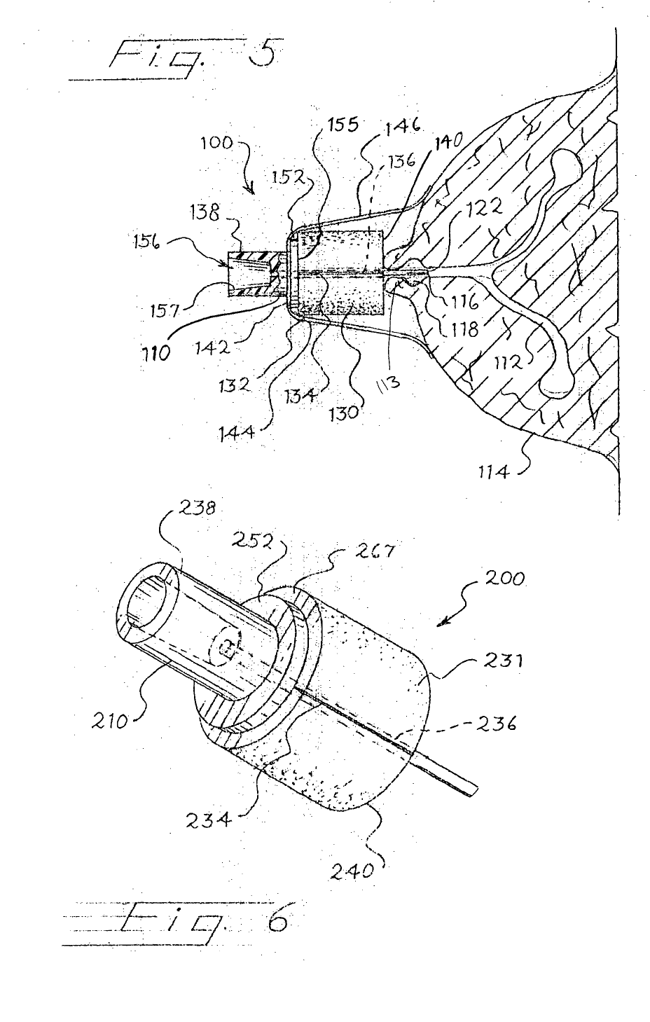

A preferred embodiment of the present invention is shown in FIG. 4. The catheter retainer 100 comprises a flexible spacer 130 and an anchor 132. Also shown in FIG. 4 is a catheter 110 having a handle 138 secured about the proximal end portion thereof.

In this embodiment, the spacer 130 is a flexible cylindrical plug, and is preferably made of a compressible elastomer, such as an open or closed cell foamed elastomer, e.g., a polyurethane foam. The spacer 130 also defines a radial slit 134, which allows the spacer 130 to be positioned about catheter 110 after the catheter distal end portion 116 has been introduced into the mammary duct, such as about intermediate portion 136. The spacer 130 may also include an adhesive layer 139 on the distal end 140 thereof which may be utilized to secure spacer 130 to the patient. When included in a kit, spacers of different sizes may be included to accommodate different patients. As described, spacer 130 is preferably a compressible material, however, this may not be necessary. If desired, the distal end portion of spacer 130 may be provided with a cavity sized to accommodate all or part of the breast nipple.

The anchor 132 is removably engageable with the catheter 110. In this embodiment, anchor 132 includes a central portion 142 and a pair of wing portions 144, 146. At least a portion of each of wing portions 144 and 146 includes an adhesive disposed on a first surface 148 thereof. Each of wing portions 144 and 146 also include a release strip 145 and 147, respectively, which is removed during use to expose the adhesive. Anchor 132 further includes a hole or aperture 150 in the central portion 142, which is configured to permit a portion of the catheter handle 138 to be extended therethough. The amount of catheter handle 138 that may be extended through hole 150 is limited by a stop such as detent 152, which may be an integrally formed ring about handle 138, and larger than aperture 150.

FIG. 5 shows the catheter retainer 100 in place during a ductal lavage procedure on a patient. The distal end portion 116 of catheter 110 is situated within mammary duct 112. In particular, open distal end 122 is situated proximate to the sinus 118 of the mammary duct to optimize the harvesting of cytological liquid. In use, spacer 130 is not positioned about an intermediate portion of catheter 110 until after it is positioned within the mammary duct 112 as desired. This is so that the operating field, namely, the nipple surface, is not obscured or impeded by the spacer as the catheter is introduced into the mammary duct, thus making the probing of the mammary duct easier.

When the spacer 130 is in place about catheter 110, which is shown partially in phantom, intermediate portion 136 of catheter 110 is nested within the radial slit 134 of spacer 130, which is biased towards a closed position. Spacer 130 is bounded on distal end 140 by the nipple 113 of breast 114, and on a proximal end 155 by detent 152. Accordingly, the spacer 130 prevents substantial movement by the catheter in a distal or proximal direction, thereby substantially maintaining the desired depth for the catheter distal end portion 116. Also, spacer 130 resists angular displacement of the catheter 110. If desired, spacer 130 may include an adhesive within slit 134 to further secure the intermediate portion 136 of catheter 110 therein. Anchor 132 is engaged with catheter 110 by guiding the aperture 150 (FIG. 4) in central portion 142 over handle 138 until the anchor central portion 142 abuts detent 152. Wing portions 144 and 146 are removably secured to the patient's breast 114 to prevent the catheter from being accidentally withdrawn from the mammary duct. Preferably, wing portions 144 and 146 are made of a flexible resilient material that may also act to provide lateral support to the catheter 110.

Preferably, handle 138 includes a through passage 156, which may define a luer taper 157, in fluid communication with catheter 110. A syringe (not shown) or other vacuum source such as a squeeze bulb may be connected to the catheter 110 by through passage 156.

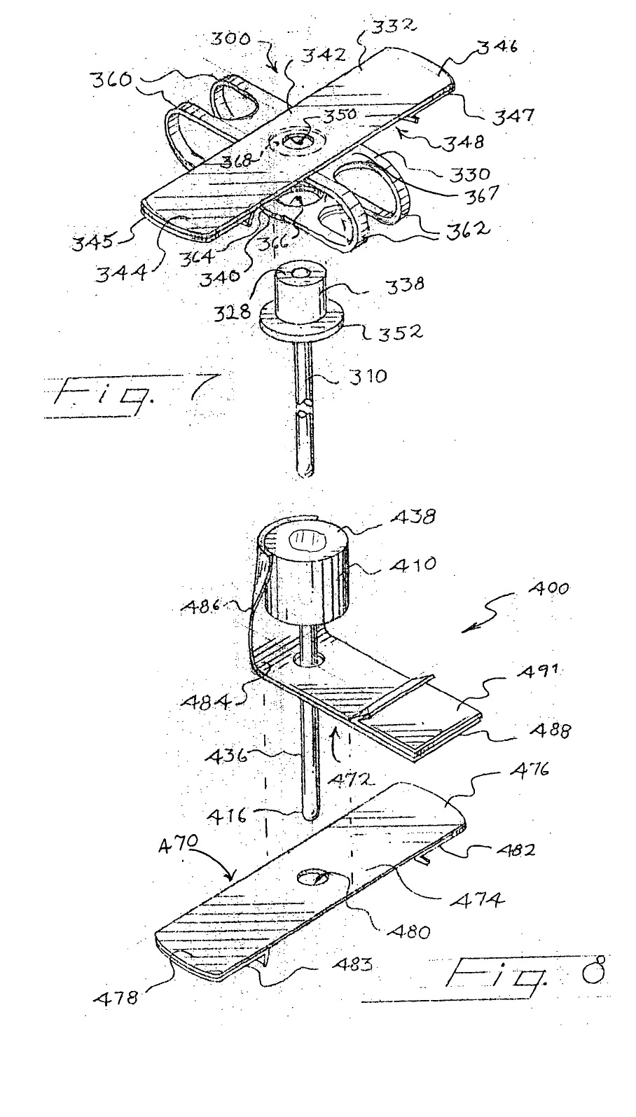

An alternate embodiment according to the present invention is shown in FIG. 6. In this particular embodiment, catheter retainer 200, spacer and anchor are integral with one another and form a spacer/anchor member 231. Spacer/anchor member 231 has a proximal end 267 and a distal end 240, and a slit 234. Spacer/anchor member 231 is made of a compressible foam, similar to the embodiment shown in FIG. 4. Catheter 210 is depicted with an intermediate portion 236 partially in phantom nested within the radial slit 234 of spacer/anchor 231, and a handle 238 having a detent 252 formed therewith. Spacer/anchor member 231 includes adhesive on proximal end 267 and distal end 240, which are covered before use by release paper members (not shown). Detent 252 is preferably of a larger diameter than the detent shown in FIG. 4 so that spacer/anchor member 231 may be securely adhered thereto by adhesive on proximal end 267. Similarly, distal end 240 is removably secured to the nipple surface with an adhesive. The catheter intermediate portion 236 may also be secured within spacer radial slit 234 with an adhesive in addition to the frictional engagement provided by the bias of spacer/anchor member 231 to a position wherein the slit 234 is closed. Typically, the resistance of such an embodiment to misaligning forces on the catheter is less than with the previous embodiment. However, in certain situations, such as if a lightweight bulb-type syringe is utilized, the stabilizing properties of the retainer 200 are sufficient.

Yet another alternate embodiment of a catheter retainer 300 according to the present invention is shown in FIG. 7. In this embodiment, spacer 330 is a spring-type member, such as leaf spring members 360 and 362. Leaf spring members 360 and 362 are unitary with one another and are connected by integral ring portion 364 at the distal end of spacer 330. Ring 364 also defines hole 366, which is configured to allow the entire catheter 310 to pass therethrough. A proximal end portion 367 of spacer 330 also defines a hole 368 which is configured to pass over at least a portion of catheter handle 338. In this particular embodiment, hole 368 is of sufficient diameter to pass over detent 352 formed on catheter handle 338. Leaf spring members 360 and 362, and spacer 330 in its entirety, is preferably formed of a resilient plastic, such as polyethylene and the like. Anchor 332 is removably engageable with the catheter 310. In this embodiment, anchor 332 includes a central portion 342 and wing portions 344, 346, each of which include an adhesive disposed on a first surface 348 thereof, as well as release strips 345 and 347, respectively, which are removed to expose the adhesive. Anchor 332 further includes a hole 350 in the central portion 342, which is configured to permit a portion of the handle 338 to be extended therethough. The amount of handle 338 that may be extended through hole 350 is limited by detent 352, which may be an integrally formed ring about handle 338 that is larger than hole 350. An adhesive may also be disposed on a distal end 340 of spacer 330 about ring 364 to further secure catheter retainer 300 to the patient.

In use, after catheter 310 is inserted into a mammary duct, spacer 330 is guided over catheter 310 and ring 364 contacts the nipple surface. The proximal end portion 328 of catheter 310 is guided through hole 368. The proximal end portion 328 of catheter 310 is also passed through hole 350 of anchor 332 such that the distal side of central portion 342 of anchor 332 abuts detent 352 as well as the proximal end 367 of spacer 330. Release strips 345 and 347 are removed and the wing portions 344 and 346 are adhered to the breast. Accordingly, spacer 330 provides support against axial movement of the catheter 310 in a distal direction, while anchor 332 resists axial movement in a proximal direction. Spacer 330 in combination with anchor 332 also provide resistance to lateral or angular movement of catheter 310.

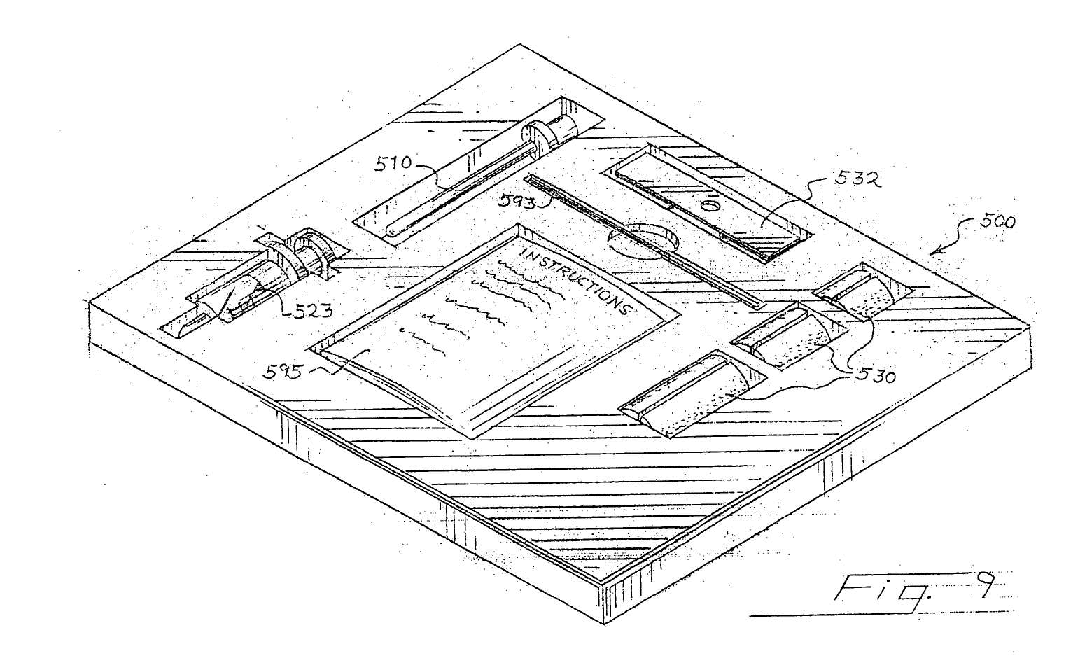

Yet another embodiment of the present invention is shown in FIG. 8. In this embodiment, catheter retainer 400 includes two flexible resilient bands 470 and 472 that in combination form both the flexible spacer and the anchor. Bands 470 and 472 may be separate or integral components, but are shown as separate parts. Band 470 includes a central portion 474 and anchor wings 476 and 478. Central portion 474 defines an aperture 480 sized to allow intermediate portion 436 of catheter 410 to pass therethrough. The distal surface of each of anchor wings 476 and 478 includes an adhesive (not shown) on which is positioned release strip 482 and 483. In use, release strips 482 and 483 are removed to expose the adhesive in order to secure the anchor wings 476 and 478 to the patient's breast. Anchor wings 476 and 478 are preferably formed of a resilient material, such as a thin flexible plastic in order to provide stabilization distally and laterally. Anchor wings 476 and 478 also have a sufficient degree of flexural rigidity to maintain the desired depth within the mammary duct for the distal end portion 416 of catheter 410. Band 472 is similar in construction to band 470, and includes a central portion 484 and securement wings 486 and 488, which have an adhesive disposed on a proximal side thereon, and release strips, such as release strip 491, positioned on the adhesive. In use, the release strips are removed and securement wings 486 and 488 are secured to catheter handle 438, such as shown with wing 486. In this embodiment catheter handle 438 is depicted as having a broadened surface to provide a larger area to which wings 486 and 488 may be adhered. As such, the position of catheter 410 relative to catheter retainer 400 is maintained. Preferably, central portion 474 and central portion 484 are matingly configured and are formed of a relatively rigid material. In order to provide for lateral stability, band 470 and band 472 are aligned perpendicular to one another. Band 470 and band 472 may be secured to one another about their respective central portions 474 and 484 through any means known in the art, such as by adhesive bonding, integrally forming the bands, clipping, hook and loop attachment, or the like.

Shown in FIG. 9 is a kit according to the present invention. Kit 500 includes a catheter 510, a plurality of flexible spacers 530 of different sizes, each of which are positionable about the catheter, and an anchor 532 engageable with the catheter 510 and removably securable to a patient's breast. Kit 500 may also include other items such as a dilator 593, a syringe 523, and instructional material 595.

The foregoing description is to be taken as illustrative, but not limiting. Still other variants within the spirit and scope of the present invention will readily present themselves to those skilled in the art.

1. A catheter retainer suitable for holding a catheter in a predetermined position within a mammary duct of a human breast, the catheter retainer comprising:
a spacer positionable about the catheter sized for receiving a portion of the catheter, and having a distal end portion for contacting the breast and a proximal end; and
an anchor operatively engageable at the proximal end portion of the spacer with a catheter received within the spacer and removably securable to the breast.

2. The catheter retainer of claim 1, wherein the spacer is flexible.

3. The catheter retainer of claim 1, wherein the spacer is a plug of a compressible elastomer.

4. The catheter retainer of claim 3, wherein the compressible elastomer is polyurethane foam.

5. The catheter retainer of claim 3, wherein the spacer defines a slit for receiving the catheter.

6. The catheter retainer of claim 1, wherein the spacer includes an adhesive on the distal end thereof for removably securing the spacer to the breast.

7. The catheter retainer of claim 1, wherein the anchor is removably engageable with the catheter.

8. The catheter retainer of claim 7, wherein the anchor defines an aperture through which a portion of the catheter is received.

9. The catheter retainer of claim 1, wherein the anchor includes an adhesive for removably securing the anchor to the breast.

10. The catheter retainer of claim 1, wherein the anchor and spacer are integral with one another.

11. The catheter retainer of claim 1, wherein an intermediate portion of the catheter is received by the spacer.

12. A kit comprising:
a catheter,
a flexible spacer for receiving a portion of the catheter and having a distal end portion and a proximal end portion;
an anchor operatively engageable with the catheter and removably securable to the breast; and
the spacer and the anchor, in concert, substantially maintaining a predetermined position of the catheter in the mammary duct.

13. The kit of claim 12, wherein the flexible spacer is a substantially cylindrical plug of a compressible elastomer.

14. The kit of claim 13, wherein the flexible spacer defines a slit for receiving an intermediate portion of the catheter.

15. The kit of claim 12, wherein the spacer includes an adhesive on the distal end thereof for removably securing the spacer to the human breast.

16. The kit of claim 12, wherein the anchor is removably engageable with the catheter.

17. The kit of claim 16, wherein the anchor defines an aperture through which a portion of the catheter can extend.

18. The kit of claim 12, wherein the anchor includes an adhesive for removably securing the anchor to the breast.

19. The kit of claim 12, wherein the catheter includes a hub.

20. The kit of claim 19, wherein the hub defines a through passage in fluid communication with the catheter.

21. The kit of claim 19, wherein the through passage terminates as a luer taper.

22. The kit of claim 12, wherein the catheter includes a stop member for limiting extension of the catheter through the aperture defined by the anchor.

23. The kit of claim 22, wherein the stop member is an annular detent.

24. The kit of claim 12, wherein the spacer is a spring member.

25. The kit of claim 24, wherein the spacer is a leaf spring.

26. The kit of claim 12, wherein the anchor and spacer are integral with one another.

27. The catheter retainer of claim 12, wherein an intermediate portion of the catheter is received by the spacer.

28. A retainer comprising suitable for use in a ductal lavage or nipple fluid aspiration procedure in association with a catheter introduced into a mammary duct of a human breast and comprising:
a spacer for receiving a portion of the catheter, and an anchor operatively engageable with the catheter and removably securable to the breast;
the spacer and the anchor, in concert, substantially maintaining the catheter at a predetermined position in the mammary duct.

29. The catheter retainer of claim 28, wherein the flexible spacer is a cylindrical plug of a compressible elastomer.

30. The catheter retainer of claim 29, wherein the flexible spacer defines a slit for receiving an intermediate portion of the catheter.

31. The catheter retainer of claim 28, wherein the spacer includes an adhesive for removably securing the spacer to the human breast.

32. The catheter retainer of claim 28, wherein the anchor is removably engageable with the catheter.

33. The catheter retainer of claim 32, wherein the anchor defines an aperture for receiving a portion of the catheter.

34. The catheter retainer of claim 28, wherein the anchor includes an adhesive for removably securing the anchor to the breast and to the catheter.

35. The catheter retainer of claim 28, wherein the anchor and spacer are integral with one another.

36. The catheter retainer of claim 28, wherein an intermediate portion of the catheter is received by the spacer.
